(19) United States
(12) Patent Application Publication
(10) Pub. No.: US 2008/0207583 A1
(43) Pub. Date: Aug. 28, 2008

(54) Process For Preparing Granulates Comprising Amoxicillin

(75) Inventor: Franz Xaver Schwarz, Worgl (AT)

Correspondence Address: SANDOZ INC, 506 CARNEFIE CENTER, PRINCETON, NJ, 08540, US

(73) Assignee: SANDOZ AG, BASEL (CH)

(21) Appl. No.: 11/813,380

(22) PCT Filed: May 1, 2006
(86) PCT No.: PCT/EP06/00053

§ 371 Date: Jul. 5, 2007

(30) Foreign Application Priority Data
Jan. 7, 2005 (SI) P200500005

(51) Int. Cl. A61K 31/43; C07D 499/48; A61P 31/04

(52) U.S. Cl. 514/197; 540/336

(57) ABSTRACT

A novel process for preparing a stable granulate, which comprises a mixture of amoxicillin trihydrate and amoxicillin sodium, by means of extrusion granulation of amoxicillin trihydrate with an aqueous solution of sodium hydroxide, sodium bicarbonate, sodium carbonate and mixture thereof or with an aqueous solution of sodium salt of (C₁₋₈) organic acid as a sodium source to form a moist extruded mass, which is granulated through a sieve and dried the obtained granulate, is disclosed. Alternatively, a novel process for preparing a stable granulate comprising pure amoxicillin sodium may be obtained according to the above extrusion granulation process as well. In a further aspect the present invention relates to a novel stable granulate comprising the mixture of amoxicillin trihydrate and amoxicillin sodium or alternatively, to a novel stable granulate comprising pure amoxicillin sodium, whenever prepared according to the above described process. Novel granulate of the present invention may be used for reconstitution with water into aqueous suspension, for parenteral injection administration, for preparing solid dosage forms, e.g. tablets, dispersible tablets, capsules, chewable tablets, and is used for treating bacterial infections in humans or animals.

### FIELD OF THE INVENTION

The present invention belongs to the field of pharmaceutical industry and relates to a novel process for preparing a stable granulate, comprising a mixture of amoxicillin trihydrate and amoxicillin sodium, by means of extrusion granulation of amoxicillin trihydrate with an aqueous solution of sodium hydroxide, sodium bicarbonate, sodium carbonate and mixture thereof or with an aqueous solution of a sodium salt of (C₁₋₈) organic acid as a sodium source, and to a method for the treatment of bacterial infection in humans or animals. Alternatively, the present invention relates to a novel process for preparing a stable granulate comprising pure amoxicillin sodium, by means of said extrusion granulation of amoxicillin trihydrate.

In a further aspect the present invention relates to the novel stable granulate comprising the mixture of amoxicillin trihydrate and amoxicillin sodium or alternatively, to the novel stable granulate comprising pure amoxicillin sodium, prepared according to above described process.

### Technical Problem

There is a constant need for preparing stable dosage forms of a combination of amoxicillin trihydrate and amoxicillin sodium or alternatively, pure amoxicillin sodium, e.g. granulate or tablets, for use in humans and animals, which may be used for reconstitution of said granulate with water into aqueous suspension, for parenteral injection administration, for preparing solid dosage forms, e.g. tablets, dispersible tablets, capsules, powders, chewable tablets.

### PRIOR ART

Amoxicillin is an antibacterial agent extensively used for the treatment of a wide range of bacterial infections, e.g. for the treatment of respiratory infections and urinary tract infections. Amoxicillin is described in The Merck Index, An Encyclopedia of Chemicals, Drugs, and Biologicals, 13^{Ed}, 2001, under monographic number 582. Amoxicillin trihydrate is the stable form of amoxicillin and is available on the market in a number of different formulations for oral administration, for example, as granulate or powders, oral suspension, hard gelatine capsules, tablets, chewable tablets, pediatric drops. Amoxicillin sodium is used in the therapy for parenteral administration. Amoxicillin in the form of hydrates and alkali salts was described for the first time in the British patent no. 1,241,844.

Patent literature describe several processes for the preparation of amoxicillin sodium salt. For example, in one of them amoxicillin trihydrate is suspending in methylene dichloride, treating with suitable amine, e.g. diethylamine or triethylamine as solubilizing agent to form amine salt, adding methanol to facilitate dissolution, dehydrating with “molecular sieve”, filtering and finally precipitating the sodium salt of amoxicillin by the addition of sodium 2-ethylhexanoate to the filtrate. On commercial scale the product is found to be contaminated by residual reagents such as triethylamine or excessive amounts of residual solvents. Drying from aqueous systems is difficult due to very high aqueous solubility of amoxicillin sodium. Furthermore, the low stability of aqueous solution of amoxicillin sodium, enhanced by its relatively high pH value, makes drying more difficult.

British patent no. 1,527,557 describes a process for the preparation of solid amoxicillin sodium by freeze-drying a solution of amoxicillin sodium in a solvent system.

British patents no. 1,576,721 and no. 2,096,599 describe a process for the preparation of solid amoxicillin sodium by spray-drying a solution of amoxicillin sodium in aqueous isopropyl alcohol or aqueous tert-butanol. When amoxicillin sodium is prepared by spray-drying or freeze-drying of an aqueous solution of amoxicillin sodium the content of degradation products, e.g. polymeric products and water content may be extremely high, which negatively influence to the stability of the product.

Patent EP-B-0 131 147 describes crystalline amoxicillin sodium and the process for its preparation. The process use large amounts of solvent mixture, e.g. methanol, methyl acetate and methylene chloride and amine, e.g. triethylamine, is used as solubilizing agent for starting amoxicillin trihydrate.

EP-B-0 596 262 describes a process for preparing sterile amoxicillin sodium from amoxicillin trihydrate in an alcohol containing solvent, in the presence of an amine, e.g. triethylamine, and sodium carboxylate, but the solvent mixture used is even more complex than that described in EP-B-0 131 147 and besides methanol and methyl acetate a further C₂₋₅ alcohol is used.

Presumably for above reason commercially available sodium amoxicillin has been obtained by spray-drying method. Amoxicillin sodium is highly water soluble antibiotic used for parenteral administration, but is significantly less stable than amoxicillin trihydrate, in particular on account of its pronounced hygroscopic nature.

All of above described processes require a high level of technical effort or cost expenditure and these disadvantages could be the reason that amoxicillin sodium is currently hardly available on the market for oral use.

Oral forms of amoxicillin sodium have been described in the patent literature, but such forms are currently not available on the market. WO 94/00112 describes an extended-release preparation containing amoxicillin sodium for oral administration for the treatment of infections caused by Heliobacter pylori in the upper gastrointestinal tract. WO 98/40054 describes an enteric coated oral dosage form comprising amoxicillin sodium for use in the treatment of Heliobacter pylori infections.

U.S. Pat. No. 6,756,057 describes that bacterial infections may be treated using high dosage regimen of amoxicillin and potassium clavulanate. Preferably, the dosage is provided by a bilayered tablet. Formulations provides a unit dosage of amoxicillin and potassium clavulanate, wherein the first immediate release phase contain all of potassium clavulanate and from 0% to 50% of the amoxicillin in the form of trihydrate and a slow release phase containing from 50% to 100% of amoxicillin in the form of crystallized sodium salt, and citric acid. Commercial drug Augmentin® XR has been marketed in the USA and also in Europe on the basis of said patent.

### DESCRIPTION OF THE INVENTION

The object of the present invention is to find a novel process for preparing a stable granulate comprising a mixture of amoxicillin trihydrate and amoxicillin sodium or alternatively, to find a novel process for preparing a stable granulate comprising pure amoxicillin sodium, wherein the exacting spray-drying or freeze-drying methods and the use of organic solvents and amine, e.g. triethylamine, used in the prior art processes for preparing amoxicillin sodium, would be omitted. The ratio of amoxicillin trihydrate to amoxicillin sodium to one another in the granulate can be adjusted with the quantity of the sodium salt source to starting amoxicillin trihydrate used according to the novel process of the invention.

In a further aspect the present invention relates to the novel stable granulate comprising the mixture of amoxicillin trihydrate and amoxicillin sodium or alternatively to the novel stable granulate comprising pure amoxicillin sodium, whenever prepared according to the above described process.

We have surprisingly and unexpectedly found that the problem known in the prior art has been solved by the novel process of the present invention, wherein amoxicillin trihydrate is extruded with an aqueous solution of sodium hydroxide, sodium bicarbonate, sodium carbonate and mixture thereof or with an aqueous solution of a sodium salt of (C₁₋₈) organic acid as a sodium source, by means of commercially available extruder to obtain granulate of the mixture of amoxicillin trihydrate and amoxicillin sodium. Amoxicillin sodium in said mixture with amoxicillin trihydrate may thus be used for oral administration.

The ratio of above sodium source to starting amoxicillin trihydrate used according to the process of the present invention for preparing said antibiotic mixture in the granulate can vary from about 0.20 to about 0.95 mol of sodium source to 1 mol of amoxicillin trihydrate.

A further aspect of the present invention provides a novel process for preparing granulate comprising pure amoxicillin sodium, wherein amoxicillin trihydrate is extruded with the aqueous solution of sodium hydroxide, sodium bicarbonate, sodium carbonate and mixture thereof or with the aqueous solution of the sodium salt of (C₁₋₈) organic acid as the sodium source by means of commercially available extruder.

The ratio of above sodium source to starting amoxicillin trihydrate used according to the process of the invention for preparing granulate comprising pure amoxicillin sodium in the granulate is suitable in approximately equivalent amounts.

The term “pure” amoxicillin sodium means amoxicillin sodium free of any residual organic solvent, e.g. methanol or methylene chloride, and free of any traces of amines, e.g. triethylamine.

The yields of extrusion granulation according to the present invention are high.

Extrusion process and extruders are known in the art, for example as described in J. Swarbrich and James C. Boylan, Encyclopedia of Pharmaceutical Technology, Vol. 5, 1992, pp 395-442.

EP-B-0 080 862 relates to water-dispersible composition for oral administration in the treatment of bacterial infections comprising amoxicillin trihydrate and potassium clavulanate and conventional excipients, wherein it contains a non-hygroscopic water-soluble binder, e.g. hydroxypropylmethylcellulose, in an amount sufficient to render said composition extrudable after addition of an organic solvent for a binder.

According to the process of the present invention the extrusion granulation of amoxicillin trihydrate with the aqueous solution of sodium hydroxide, sodium bicarbonate, sodium carbonate and mixture thereof or with an aqueous solution of sodium salt of (C₁₋₈) organic acid as the sodium source is performed at appropriate extrusion temperatures, e.g. between 0° C. to 60° C., preferably between 10° C. to 40° C. to obtain moist extruded mass. The obtained moist extruded mass is granulated through a sieve, subsequently dried the obtained granulate, which is optionally re-dryed to obtain the dry granulate of the mixture of amoxicillin trihydrate and amoxicillin sodium in the form of a mixture of crystal form, amorphous form and agglomerates. Said antibiotic mixture form has not been disclosed in the prior art and is therefore novel.

Alternatively, extrusion granulation of amoxicillin trihydrate suitably in approximately equivalent amount to sodium source according to above described process provides granulate comprising pure amoxicillin sodium.

As the sodium salt of (C₁₋₈) organic acid for extrusion granulation process may be used the sodium salt of acetic acid, propionic acid, butyric acid, valeric acid, caproic acid, caprilic acid and capric acid, preferably sodium salt of 2-ethyl hexanoate (sodium salt of 2-ethyl caproic acid).

Mesh size of the sieve used in the extrusion granulation process is in the range between 0.5 mm to 4.0 mm, preferably in the range from 1 mm to 2 mm, most preferably 1.25 mm.

Granulate comprising the mixture of amoxicillin trihydrate and amoxicillin sodium may be prepared according to the process of the present invention, comprising the following steps:
a. extruding amoxicillin trihydrate with an aqueous solution of sodium hydroxide, sodium bicarbonate, sodium carbonate and mixture thereof or with an aqueous solution of sodium salt of (C₁₋₈) organic acid as a sodium source to form a moist extruded mass,
b. granulating moist extruded mass through a sieve,
c. drying the obtained granulate, and
d. optionally re-drying granulate to obtain the dry granulate of the mixture of amoxicillin trihydrate and amoxicillin sodium

Granulate comprising pure amoxicillin sodium may be prepared according to the above steps but using in step a. amoxicillin trihydrate and sodium salt source suitable in approximately equivalent amounts.

Granulate comprising the mixture of amoxicillin trihydrate and amoxicillin sodium or alternatively, granulate comprising pure amoxicillin sodium obtained according to the process of the present invention is stable and may be presented in protective packages such as glass bottles, aluminium foil sachets containing multiple doses, or in single dose sachets, or into other suitable container protected from moisture. Granulate according to the present invention is free of any residual organic solvent, e.g. methanol or methylene chloride, and free of residual reagents such as triethylamine or other amines which were used in the prior art for preparing amoxicillin sodium.

A preferred mean particle size of the granules is in the range between 200 μm to 3000 μm, particularly between 800 μm to 1500 μm.

The dried extrudate (granulate) may be levelled as required through a more or less coarse sieve or ground with a mill. In this way powders and granulate comprising the mixture of amoxicillin trihydrate and amoxicillin sodium or alternatively, granulate and powders comprising pure amoxicillin sodium with different particle distribution can be produced.

Granulate of the present invention can also be produced discontinuously by granulation in a granulator, e.g. ribbon blenders Stephan, Disona, Collte Gral, or in a fluidised bed granulator by spray granulation, e.g. Glatt Hüttlin.

Granulate according to the invention may be directly tabletted after the addition of conventional tabletting adjuvants, e.g. flow-regulating agents, lubricants and tabletting auxiliaries.

Granular product obtained according to the present invention may be used at a range of unit doses, for example between 1 g to 5 g of the mixture of amoxicillin trihydrate and amoxicillin sodium. Granulate comprising pure amoxicillin sodium may be used for oral administration or for parenteral administration.

The invention also provides granulate comprising the mixture of amoxicillin trihydrate and amoxicillin sodium or alternatively, granulate comprising pure amoxicillin sodium, for reconstitution with water into an aqueous suspension, for use in the treatment of bacterial infections.

The invention also provides a method of treatment of bacterial infections in humans and animals which comprises the administration of a therapeutically effective amount of the mixture of amoxicillin trihydrate and amoxicillin sodium containing in the granulate or alternatively, a therapeutically effective amount of pure amoxicillin sodium containing in the granulate.

The invention is illustrated but in no way limited by the following Examples:

All examples use extrusion granulation technique on the following equipment under the conditions stated, moist granulated through a 2 mm sieve and dried in the specific fluidised bed equipment. Optionally, re-drying can take place by introducing absolutely dry air through the granulate bed at room temperature.

Extruder: Theysohn TSK N20/20D—Maschinenbau/Salzgitter (Germany)

| | |
|---|---|
| Addition of amoxycillin trihydrate powder | 80-125 g/min |
| Extruding liquid rate | 15-30 g/min |

Drying:

Fluidised bed dryer Glatt GPC 2

| | | |
|---|---|---|
| Incoming temperature | 85° | C. |
| Material temperature at the end of drying | 45-55° | C. |

Re-drying (optional):

| | |
|---|---|
| Drying air | <5% relative air humidity at 25° C. |
| Duration | 12-72 h |

### EXAMPLE 1

### Granulate Comprising Pure Amoxicillin Sodium

| Component | weight (g) |
|---|---|
| Amoxicillin trihydrate | 1000.0 |

Solution for extrusion granulation:

| Component | weight (g) |
|---|---|
| NaOH | 95.4 |
| water | 238.4 |

Amoxicillin trihydrate is extruded with the aqueous solution of sodium hydroxide under above described conditions, sieved and dried to obtain 923.6 g granulate comprising pure amoxicillin sodium.

### EXAMPLE 2

### Granulate Comprising the Mixture of Amoxicillin Trihydrate and Amoxicillin Sodium

| Component | weight (g) |
|---|---|
| Amoxicillin trihydrate | 1000.0 |

Solution for extrusion granulation:

| Component | weight (g) |
|---|---|
| NaOH | 47.7 |
| water | 220.0 |

Amoxicillin trihydrate is extruded with the aqueous solution of sodium hydroxide under above described conditions, sieved and dried to obtain granulate comprising the mixture of 500.0 g amoxicillin trihydrate and 461.8 g of amoxicillin sodium.

### EXAMPLE 3

### Granulate Comprising the Mixture of Amoxicillin Trihydrate and Amoxicillin Sodium

| Component | weight (g) |
|---|---|
| Amoxicillin trihydrate | 1000.0 |

Solution for extrusion granulation:

| Component | weight (g) |
|---|---|
| Na₂CO₃ | 63.2 |
| water | 230.0 |

Amoxicillin trihydrate is extruded with the aqueous solution of sodium carbonate under above described conditions, sieved and dried to obtain granulate comprising the mixture of 500.0 g of amoxicillin trihydrate and 461.8 g of amoxicillin sodium.

### EXAMPLE 4

### Granulate Comprising Pure Amoxicillin Sodium

| Component | weight (g) |
|---|---|
| Amoxicillin trihydrate | 1000.0 |
| Na₂CO₃ | 126.3 |

A mixture of amoxicillin trihydrate and sodium carbonate is extruded with 220.0 g of water under above described conditions, sieved and dried to obtain 923.6 g of granulate comprising pure amoxicillin sodium.

### EXAMPLE 5

### Granulate Comprising the Mixture of Amoxicillin Trihydrate and Sodium Amoxicillin

| Component | weight (g) |
|---|---|
| Amoxicillin trihydrate | 1000.0 |
| NaHCO₃ | 70.1 |
| water | 220.0 |

A mixture of 1000.0 g amoxicillin trihydrate, 70.1 g of sodium bicarbonate and 220.0 g of water is granulated for 3 minutes in the Stephan mixer, then sieved and dried according to above conditions to obtain granulate comprising the mixture of 650.0 g amoxicillin trihydrate and 323.2 g of amoxicillin sodium.

1. A process for preparing a stable granulate of a mixture comprising amoxicillin trihydrate and amoxicillin sodium, the process comprising the following steps:
a. extruding amoxicillin trihydrate with an aqueous solution of sodium hydroxide, sodium bicarbonate, sodium carbonate or a mixture thereof or with an aqueous solution of a sodium salt of C₁₋₈ organic acid as a sodium source to form a moist extruded mass,
b. granulating the moist extruded mass through a sieve, yielding a granulate comprising a mixture of amoxicillin trihydrate and amoxicillin sodium,
c. drying the granulate, and
d. optionally re-drying the granulate.

2. The process according to claim 1, wherein the C₁₋₈ organic acid is selected from the group consisting of acetic acid, propionic acid, butyric acid, valeric acid, caproic acid, caprilic acid and capric acid.

3. The process according to claim 1, wherein the sodium salt of C₁₋₈ organic acid is sodium 2-ethyl hexanoate.

4. The process according to claim 1, wherein the sodium source is present in a ratio of between about 0.20 mol to about 0.95 mol to 1 mol of amoxicillin trihydrate.

5. The process according to claim 1, wherein the process has an extrusion temperature between 0° C. to 60° C.

6. The process according to claim 5, wherein the extrusion temperature is between 10° C. to 40° C.

7. The process according to claim 1, wherein the granulate has a particle size of 200 μm to 3000 μm.

8. The process according to claim 7, wherein the particle size is in the range of 800 μm to 1500 μm.

9. The process according to claim 1, wherein the granulate comprises a mixture of amoxicillin trihydrate and amoxicillin sodium in the form of a mixture of crystal form, amorphous form and agglomerates.

10. The process of claim 9, wherein the granulate is substantially free of any residual organic solvent.

11. The process of claim 9 further comprising the step of reconstituting the granulate into an aqueous suspension prior to use.

12. The process of claim 9, wherein the granulate is provided in glass bottles, unit dose sachets or into other suitable container protected from moisture.

13. A method of treating bacterial infections in humans or animals by administering a therapeutically effective amount of the mixture of amoxicillin trihydrate and amoxicillin sodium containing in the granulate prepared according to claim 1.

14. A method of using the granulate produced by the process of claim 9 to treat bacterial infections in humans or animals.

15. A process for preparing a stable granulate comprising substantially pure amoxicillin sodium, the process comprising the following steps:
a. extruding amoxicillin trihydrate with an aqueous solution of sodium hydroxide, sodium bicarbonate, sodium carbonate or a mixture thereof or with an aqueous solution of a sodium salt of C₁₋₈ organic acid as a sodium source suitably in approximately equivalent amounts to form a moist extruded mass,
b. granulating the moist extruded mass through a sieve, yielding a granulate of substantially pure amoxicillin sodium,
c. drying the granulate, and
d. optionally re-drying the granulate to obtain the dry granulate of pure amoxicillin.

16. The granulate prepared according to the process of claim 15, wherein the granulate is substantially free of any residual organic solvent and substantially free of any traces of amine.
